(19) United States
(12) Patent Application Publication
(10) Pub. No.: US 2026/0316985 A1
(43) Pub. Date: Oct. 15, 2026

(54) SYRINGE NEST FOR IMPROVED ASEPTIC FILLING AND STOPPERING

(71) Applicant: SIO2 MEDICAL PRODUCTS, INC., Auburm, AL (US)

(72) Inventors: Aaron Hill, Auburn, AL (US); Brian Fitzpatrick, Auburn, AL (US)

(21) Appl. No.: 19/116,564

(22) PCT Filed: Sep. 29, 2023
(86) PCT No.: PCT/US2023/075473

§ 371 Date: Mar. 28, 2025

Related U.S. Application Data
(60) Provisional application No. 63/411,381, filed on Sep. 29, 2022.

(51) Int. Cl. B65B 43/54; A61M 5/00; A61M 5/50; B65B 3/00; B65D 25/10; B65D 85/30

(52) U.S. Cl. CPC B65B 43/54; A61M 5/002; A61M 5/008; A61M 5/5086; B65B 3/006; B65D 25/108; B65D 85/30; A61M 2207/00; B65B 2220/14

(57) ABSTRACT

A syringe nest includes a substantially planar base and a plurality of syringe receptacles. Each receptacle includes a cylindrical wall defining an upper opening. An inner surface of the cylindrical wall has a diameter that is larger than the diameter of the main body portion but smaller than the diameter of the rear flange of the syringe barrel. One or more upward-extending ribs are positioned around the upper opening of each receptacle and limit radial movement of a syringe barrel within the receptacle through contact with the rear flange. Centering of the syringe barrels in the receptacles in this manner results in an improved process for preparing pre-filled syringes, and specifically improved filling and stoppering steps.

TThis application claims priority to U.S. provisional patent application No. 63/411,381, filed on Sep. 29, 2022, the entirety of which is incorporated by reference herein.

### FIELD OF THE INVENTION

Embodiments of the present disclosure relate to syringe nests, in particular those configured to hold a plurality of syringe barrels during the preparation of pre-filled syringes, and specifically during the filling and/or stoppering steps.

### SUMMARY OF THE INVENTION

Pre-filled syringes are increasingly important in the pharmaceutical industry. In order to fill and seal, or stopper, the filled syringes, syringe barrels having a main body portion, a front dispensing portion, and a rear flange are typically provided in a tray, generally known as a “syringe nest”. A syringe nest is configured to hold a plurality of syringe barrels. For example, some syringe nests may be configured to hold 100 syringes, e.g. contain 10 rows of 10 syringe barrel receptacles.

The front dispensing portion of the syringe barrel, which most commonly comprises either a staked needle or a Luer tip configured to receive a needle attachment by way of a Luer connection, is sealed by a removable tip cap, e.g. a rigid needle shield in the case of a staked needle or a tip cover in the case of a Luer tip. Conventionally, the diameter of the tip cap may be similar to the diameter of the main body portion of the syringe barrel. The rear flange, which is often (but not always) circular in cross-section, has a diameter that is greater than the diameter of the main body portion of the syringe barrel.

A syringe nest comprises a substantially planar base and a plurality of syringe receptacles, i.e. wells, defined in the base. Each of the receptacles includes a cylindrical wall defining an upper opening and a lower opening. The inner diameter of the cylindrical wall is larger than the diameter of the main body portion of the syringe barrel (and the diameter of the tip cap) but smaller than the diameter of the rear flange, so that the syringe barrel may be inserted downward into the upper opening until the rear flange is seated on a top surface of the cylindrical wall. Typically, the front dispensing portion, sealed by the removable tip cap, and some amount of the main body portion of the syringe barrel extend out of the lower opening of the receptacle. Because the removable tip cap conventionally has a diameter that is similar to the diameter of the main body portion of the syringe barrel, each receptacle may be configured such that the inner diameter of the cylindrical wall is only slightly larger than the diameter of the main body portion of the syringe barrel. Radial, i.e. lateral, movement of the syringe barrel within the receptacle is thus limited.

The syringe barrels are typically filled via an aseptic filling machine. During the aseptic filling process, the syringe nest containing a plurality of syringe barrels is aligned with a fixture comprising a plurality of filling needles (the number of filling needles corresponding with the number of syringe barrels held by the syringe nest, i.e. with the number of receptacles in the nest). The filling needles are then inserted into the lumens of the syringe barrels through the rear openings. A predetermined volume of an injectable medical solution is deposited into the lumen and then the filling needles are removed.

After the syringe barrels are filled, the rear openings of the lumens are stoppered in order to seal off the fluid-containing lumen prior to the filled syringe barrel leaving the aseptic environment. To do so, the syringe nest is aligned with a plunger nest, also sometimes referred to as a stopper nest. The plunger nest contains a plurality of plungers, i.e. stoppers (the number of plungers corresponding with the number of syringe barrels held by the syringe nest, i.e. with the number of receptacles in the syringe nest). The syringe nest and plunger nest are also both aligned with a fixture comprising a plurality of plunger insertion rods, with the plunger nest being positioned directly between the syringe nest and the plunger insertion rods. A vacuum is achieved in the aseptic chamber to remove air that could prevent the installation of the plunger, i.e. stopper. The plunger insertion rods, also sometimes referred to as needles, are then deployed into contact with the plurality of plungers so as to push the plungers out of the plunger nest and into the lumens of the syringe barrels until a predetermined position along the axis of the syringe barrels is reached.

The filling and stoppering processes are typically fully automated, with the movement of parts being controlled through one or more central processors.

Once the syringe barrel has been filled and stoppered, it can be referred to as a pre-filled syringe. The syringe nest containing the plurality of pre-filled syringes is typically inserted into a tub. The tub typically comprises a shoulder on which the substantially planar base of the syringe nest is seated, thereby placing the plurality of pre-filled syringes within the interior space defined by the sidewalls of the tub. A cover is then sealed to the tub to enclose the syringe nest and the pre-filled syringes therein.

In embodiments of the present invention, it may be desirable to utilize a tamper evident tip cap. In this way, a pre-filled syringe would immediately indicate to a medical professional if the tip cap had been previously removed.

A tamper evident tip cap, however, may have a diameter that is significantly greater than the diameter of the main body portion of the syringe barrel. Because the syringe barrels are inserted into the receptacles of the syringe nest dispensing end first, the inner diameter of the cylindrical wall of each receptacle must be large enough to accommodate the increased diameter of the tip cap, i.e. the inner diameter of the cylindrical wall of each receptacle must be at least slightly larger than the diameter of the tip cap. The result is a gap between the main body portion of the syringe barrel and the inner surface of the cylindrical wall, which allows for radial movement of the syringe barrel within the receptacle.

During the filling of the syringe barrels, the filler needles must be aligned with the rear openings of the syringe barrels held by the syringe nest. Similarly, during the subsequent stoppering of the syringe barrels, the plungers must be aligned with the rear openings of the syringe barrels held by the syringe nest. In order to prevent complications arising from improper alignment, it has presently been determined that radial movement of the syringe barrels within the syringe nest should be reduced.

Embodiments of the present disclosure are directed to a syringe nest comprising one or more ribs positioned around the upper opening of each receptacle, the one or more ribs being configured to limit radial movement of a syringe barrel within the receptacle through contact between the one or more ribs and an outer surface of the rear flange of the syringe barrel.

Some embodiments of the present disclosure are directed to a syringe nest that includes a substantially planar base and a plurality of receptacles, each of the receptacles being sized and configured to receive a syringe barrel having a main body portion, a front dispensing portion, and a rear flange. Each receptacle includes a cylindrical wall that defines an upper opening. The diameter of each receptacle, measured from an inner surface of the cylindrical wall, is larger than the diameter of the main body portion of the syringe barrel but smaller than the diameter of the rear flange of the syringe barrel which it is configured to receive. One or more upward-extending ribs are positioned around the upper opening of each receptacle. The one or more ribs are configured to limit radial movement of a syringe barrel within the receptacle through contact between an inner surface of the one or more ribs and an outer surface of the syringe flange. By limiting radial movement, the one or more ribs are configured to maintain each of the plurality of syringe barrels substantially centered within the receptacle.

Embodiments of the present disclosure are also directed to a syringe nest, such as that described above, and a plurality of syringe barrels positioned within the plurality of receptacles. Some embodiments, for instance, are directed to (a) a syringe nest that includes a substantially planar base and a plurality of receptacles and (b) a plurality of syringe barrels, each syringe barrel being removably positioned within one of the plurality of receptacles. Each syringe barrel includes a main body portion, a front dispensing portion, and a rear flange. The diameter of each receptacle, measured from an inner surface of the cylindrical wall, is larger than the diameter of the main body portion of the syringe barrel but smaller than the diameter of the rear flange of the syringe barrel positioned therein. The syringe nest further includes one or more upward-extending ribs positioned around an upper opening of each of the plurality of receptacles. The one or more ribs limit radial movement of the syringe barrel positioned within the receptacle through contact between an inner surface of the one or more ribs and an outer surface of the syringe flange. By limiting radial movement in this manner, the one or more ribs maintains each of the plurality of syringe barrels substantially centered within the receptacle.

In some embodiments, the one or more ribs may be a single rib that extends around the circumference of the upper opening of the receptacle. Alternatively, in some embodiments, the one or more ribs may include two or more ribs, each of which extends around a portion of the circumference of the upper opening of the receptacle.

In some embodiments, at least a portion of the cylindrical wall of each of the receptacles may extend upward from the substantially planar base. In such an embodiment, the one or more ribs may extend upward from a top surface of the cylindrical wall. Alternatively, in some embodiments, the upper opening of each of the receptacles may be substantially planar with an upper surface of the substantially planar base. In such an embodiment, the one or more ribs may extend upward from the upper surface of the substantially planar base.

In some embodiments, the cylindrical wall of each receptacle may also define a lower opening.

In some embodiments, the one or more ribs may have an inner surface that is configured to be positioned 1.5 mm or less, alternatively 1.25 mm or less, alternatively 1.0 mm or less, alternatively 0.75 mm or less, alternatively 0.50 mm or less from the outer surface of the flange of a syringe barrel that the associated receptacle is configured to receive, when the syringe barrel is centered within the receptacle. For some embodiments that include a plurality of syringe barrels, the one or more ribs may have an inner surface that is positioned 1.5 mm or less, alternatively 1.25 mm or less, alternatively 1.0 mm or less, alternatively 0.75 mm or less, alternatively 0.50 mm or less from the flange of the syringe barrel positioned within the associated receptacle, when the syringe barrel is centered within the receptacle.

In any embodiment, the one or more ribs may be positioned at a radial distance from the inner surface of the cylindrical wall, i.e. from the periphery of the upper opening of the receptacle, selected to provide a desired limitation on movement of a syringe barrel that the receptacle is configured to receive or receives. In some embodiments, an inner surface of the one or more ribs may be positioned between about 0.50 mm and about 2.0 mm from the upper opening of the receptacle (i.e., the inner surface of the cylindrical wall), alternatively between about 0.75 mm and about 1.75 mm from the upper opening of the receptacle, alternatively between about 1.0 mm and about 1.5 mm from the upper opening of the receptacle.

In some embodiments, each of the one or more ribs may have a chamfered surface that is configured to direct the flange of a syringe barrel toward the upper opening of the receptacle during insertion of the syringe barrel into the receptacle.

In some embodiments, the diameter of the inner surface of the cylindrical wall may be at least 3 mm larger than the diameter of the main body portion of the syringe barrel that the associated receptacle receives, alternatively at least 4 mm larger than the diameter of the main body portion of the syringe barrel, alternatively at least 5 mm larger than the diameter of the main body portion of the syringe barrel.

In some embodiments, the syringe barrel may include a staked needle and a removable needle shield. In some embodiments, the needle shield may be a tamper-evident needle shield. In some embodiments, the needle shield may have a diameter that is greater than the diameter of the main body portion of the syringe barrel and less than the diameter of the rear flange of the syringe barrel. Alternatively, in some embodiments, the syringe barrel may include a Luer tip and a removable tip cover. In some embodiments, the tip cover may be a tamper-evident tip cover. In some embodiments, the tip cover may have a diameter that is greater than the diameter of the main body portion of the syringe barrel and less than the diameter of the rear flange of the syringe barrel.

In some embodiments, each of the plurality of syringe barrels may have a volume of 1 mL or less, optionally less than 1 mL, optionally 0.75 mL or less, optionally less than 0.75 mL, optionally 0.5 mL or less, optionally the syringe barrels may have a volume of about 0.5 mL.

The syringe nest of any embodiment may be made of any desirable material. In some embodiments, the syringe nest may be made of plastic. For instance, the syringe nest may be made of polypropylene. In any embodiment, the syringe nest may also be injection molded.

The syringe nest of any embodiment may have a desired number of receptacles. In some embodiments, the syringe nest may have between fifty and one hundred receptacles.

Embodiments of the present disclosure are also directed to a nested set of syringe barrels that includes (a) the syringe nest of any embodiment described herein and (b) a plurality of syringe barrels, each of the plurality of syringe barrels being received within one of the plurality of receptacles of the syringe nest. Each of the syringe barrels may include a removable needle shield or a removable tip cap sealing a dispensing opening, optionally a tamper-evident removable needle shield or removable tip cap.

Embodiments of the present disclosure are also directed to a nested set of pre-filled syringes that includes (a) the syringe nest of any embodiment described herein and (b) a plurality of pre-filled syringes, each of the plurality of pre-filled syringes having a syringe barrel received within one of the plurality of receptacles of the syringe nest. Each of the pre-filled syringes may include a removable needle shield or a removable tip cap sealing a dispensing opening, optionally a tamper-evident removable needle shield or removable tip cap, an injectable medical solution in a lumen of the syringe barrel, and a plunger sealing a rear opening of the lumen.

Embodiments of the present disclosure are also directed to a package of pre-filled syringes that includes (a) the syringe nest of any embodiment described herein and (b) a plurality of pre-filled syringes, each of the plurality of pre-filled syringes having a syringe barrel received within one of the plurality of receptacles of the syringe nest, (c) a tub comprising a shoulder on which the syringe nest is seated such that the pre-filled syringes are positioned within an interior of the tub defined by the tub sidewalls, and (d) a cover sealed to the tub to enclose the pre-filled syringes. Each of the pre-filled syringes may include a removable needle shield or a removable tip cap sealing a dispensing opening, optionally a tamper-evident removable needle shield or removable tip cap, an injectable medical solution in a lumen of the syringe barrel, and a plunger sealing a rear opening of the lumen. In some embodiments, the cover may be a flexible membrane.

Embodiments of the present disclosure are also directed to a method of preparing a plurality of pre-filled syringes, which includes a stoppering step in which a plurality of plungers are inserted into the lumens of a plurality of syringe barrels. In the stoppering step, a plurality of syringe barrels are held in the syringe nest of any embodiment described herein and a plurality of plungers are held in a plunger nest. The plunger nest is then aligned with the syringe nest, and a fixture that includes a plurality of plunger insertion rods pushes the plurality of plungers out of the plunger nest and a desired distance into the lumens of the syringe barrels. The ribs of the syringe nest may ensure that each of the plurality of syringe barrels is substantially centered in a receptacle of the syringe nest during the stoppering step.

Embodiments of the present disclosure are also directed to a method of preparing a plurality of pre-filled syringes, which includes a filling step in which a medical solution is injected into the lumens of a plurality of syringe barrels. In the filling step, a plurality of syringe barrels are held in the syringe nest of any embodiment described herein. A plurality of filling needles are then inserted into the lumens of the syringe barrels, and an injectable medical solution is injected from the filling needles into the lumens of the plurality of syringe barrels. The ribs of the syringe nest may ensure that each of the plurality of syringe barrels is substantially centered in a receptacle of the syringe nest during the filling step.

Embodiments of the present disclosure are also directed to a method of preparing a plurality of pre-filled syringes that includes a stoppering step such as that described above and a filling step such as that described above. The ribs of the syringe nest may ensure that each of the plurality of syringe barrels is substantially centered in a receptacle of the syringe nest during both the stoppering step and the filling step.

As used herein, a syringe barrel being substantially centered in a receptacle means that a syringe barrel is positioned within a receptacle such that a filling needle and/or plunger aligned with the center of the receptacle will enter the rear opening of the syringe barrel.

For purposes of the present disclosure, the syringe barrels are shown and described as having rear flanges that are circular in cross-section. However, it should be understood that the concepts described herein are equally applicable to syringe barrels having rear flanges that are not circular in cross-section.

Though the present disclosure refers to tamper-evident tip caps as having diameters that are significantly larger than the main body portion of the syringe barrels to which they are attached, it should be recognized that any tip cap—whether tamper-evident or not—may have a diameter that is significantly larger than the main body portion of the syringe barrels so as to benefit from the use of an embodiment of a syringe nest described herein to reduce radial movement of the syringe barrels within the nest.

### BRIEF DESCRIPTION OF THE DRAWINGS

FIG. 1 is a perspective view of an embodiment of a syringe nest having a plurality of pre-filled syringes received therein and a tub configured to hold the syringe nest.

FIG. 2 is a perspective cross-sectional view of a portion of a syringe nest in accordance with an embodiment disclosed herein.

FIG. 3 is a side elevation view, in cross section, of a well (i.e. receptacle) of a syringe nest in accordance with an embodiment disclosed herein, showing an example syringe barrel received therein.

FIG. 4 is a perspective view of a system for stoppering a plurality of syringe barrels in accordance with an embodiment disclosed herein.

FIG. 5 is a side elevation view, in cross section, of the system for stoppering a plurality of syringe barrels in accordance with an embodiment disclosed herein.

FIG. 6 is a top plan view of a syringe nest in accordance with an embodiment disclosed herein.

FIG. 7 is a side elevation view, in cross section, of a well (i.e. receptacle) of a syringe nest in accordance with an embodiment disclosed herein.

FIG. 8 is a top plan view of an embodiment of a receptacle, showing the radial movement of a syringe barrel within the receptacle that may be permitted absent the one or more ribs disclosed herein.

FIG. 9 is a perspective view of an embodiment of a 0.5 ml syringe barrel having a tamper-evident tip cap.

FIG. 10 is a side elevation view, in cross section, of a well (i.e. receptacle) of a syringe nest in accordance with an embodiment disclosed herein, showing the gap between a main body portion of the syringe barrel and the inner surface of the cylindrical wall of the receptacle when the syringe barrel is centered in the receptacle.

FIG. 11 is a side elevation view, in cross section, of a portion of a well (i.e. receptacle) of a syringe nest in accordance with an embodiment disclosed herein, showing the distance between an outer surface of the flange of the syringe barrel and the inner surface of the one or more ribs when the syringe barrel is centered in the receptacle.

FIG. 12 is a side elevation view, in cross section, of a portion of the system for stoppering a plurality of syringe barrels in accordance with an embodiment disclosed herein, showing an alignment of a well of a plunger nest with a well of a syringe nest in accordance with an embodiment disclosed herein and prior to insertion of the plunger into the lumen of the syringe barrel.

### DETAILED DESCRIPTION

The present invention will now be described more fully, with reference to the accompanying drawings, in which several embodiments are shown. This invention can, however, be embodied in many different forms and should not be construed as limited to the embodiments set forth here. Rather, these embodiments are examples of the invention, which has the full scope indicated by the language of the claims. Like numbers refer to like or corresponding elements throughout. The following disclosure relates to all embodiments unless specifically limited to a certain embodiment.

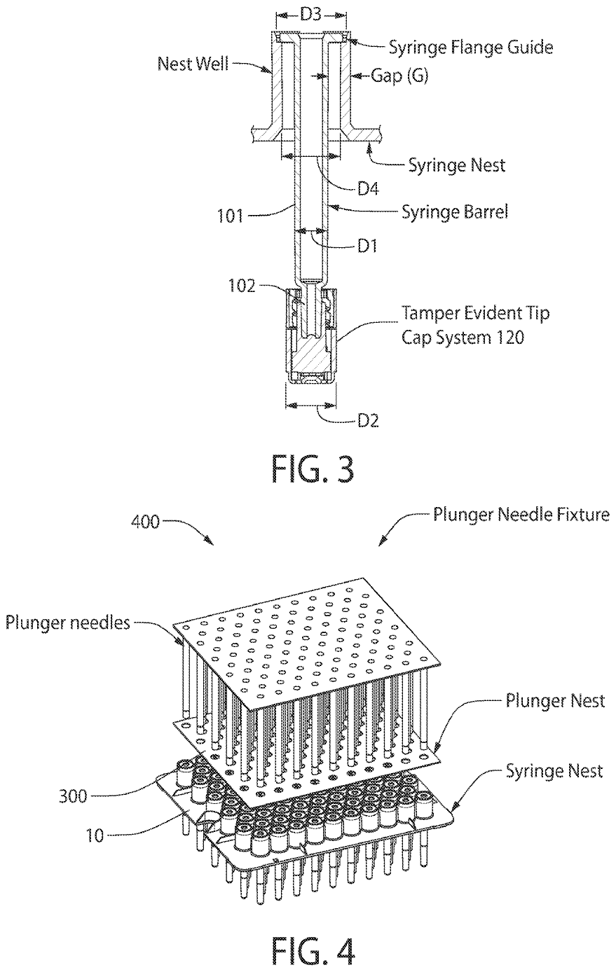

Embodiments of the present disclosure are directed to a syringe nest (either on its own or in combination with a plurality of syringe barrels or pre-filled syringes contained therein) that is configured to limit radial movement of a syringe barrel received within a well of the syringe nest. An example of such a syringe nest 10 is shown in FIGS. 2-3.

Before describing the syringe nest 10, however, a brief description of a syringe barrel, an example of which is also shown in FIGS. 2-3, is needed. A syringe barrel 100 comprises a main body portion 101, a front dispensing portion 102, and a rear flange 103. The cylindrical side wall of the syringe barrel 100 defines a central lumen 104, which prior to stoppering is accessible through a rear opening 105. Before being placed into a syringe nest 10, the front dispensing portion 102 is provided with a removable tip cap 120.

The removable tip cap 120 may take on any of a number of configurations, including for example a rigid needle shield (e.g. where the syringe is a staked needle syringe) or a tip cover (e.g. where the syringe has a Luer-compatible tip). Additionally, the removable tip cap 120 may, in some embodiments, comprise a tamper-evident component.

The main body portion 101 of the syringe barrel has a first diameter, identified in FIG. 3 as D1. The front dispensing portion 102, with the attached tip cap 120, has a second diameter, identified in FIG. 3 as D2. The rear flange 103 has a third diameter, identified in FIG. 3 as D3. As shown in FIG. 3, the diameter of the rear flange, D3, is larger than the diameter of the main body portion, D1, and the diameter of the front dispensing portion with the attached tip cap, D2, may be less than the diameter of the rear flange, D3, but significantly greater than (in that it gives rise to the radial movement problem described herein) the diameter of the main body portion, D1.

In some embodiments, the syringe barrel may comprise a small volume lumen. For instance, in some embodiments, the syringe barrel may have a volume of 1 mL or less, alternatively less than 1 mL, alternatively 0.75 mL or less, alternatively less than 0.75 mL, alternatively 0.5 mL or less, alternatively 1 mL or 0.5 mL, alternatively 0.5 mL. In some embodiments, the pre-filled syringe may have a fill volume of an injectable solution of about 300 microliters (0.3 mL). An example of a 0.5 ml syringe barrel 100 having a relatively high-diameter tamper-evident tip cap 120 is shown in FIG. 9.

In some embodiments, the syringe barrel 100 may have a main body portion 101 diameter, D1, that is between about seven and about nine millimeters, alternatively between about seven and about eight millimeters. In some embodiments, the syringe barrel 100 may have a tip cap 120 diameter, D2, that is between about ten and about thirteen millimeters, alternatively between about eleven and about twelve millimeters. In some embodiments, the syringe barrel 100 may have a flange 103 diameter, D3, that is between about twelve and about fifteen millimeters, alternatively between about thirteen and about fourteen millimeters.

Turning back to the syringe nest, an embodiment of the syringe nest 10 disclosed herein may include a planar or substantially planar base 11 and a plurality of syringe receptacles 12, i.e. wells, defined in the base. Each syringe receptacle 12 includes a cylindrical wall 13 having an inner surface 14 which spans between and defines an upper opening 15 and a lower opening 17. Each syringe receptacle 12 is sized and configured to receive a syringe barrel 100.

For instance, the cylindrical wall 13 has an inner surface 14 having a diameter, identified in FIG. 3 as D4, that is large enough to receive the main body portion 101 of the syringe barrel and the front dispensing portion 102 of the syringe barrel with the attached tip cap 120. Where, as in the illustrated embodiment, the diameter of the front dispensing portion of the syringe barrel with the attached tip cap, D2, is greater than the diameter of the main body portion of the syringe barrel, D1, the result is a gap, G, between the main body portion 101 of the syringe barrel and the inner surface 14 of the cylindrical wall 13.

The inner surface 14 of the cylindrical wall 13, however, has a diameter, D4, that is smaller than the diameter of the rear flange 103 of the syringe barrel, D3. In this way, the rear flange 103 of the syringe barrel is seated on a top surface 16 of the cylindrical wall, thereby preventing the syringe barrel 100 from sliding completely through the receptacle 12. In the illustrated embodiment, the cylindrical wall 12 extends upward from the base 11 to the top surface 16. In other (non-illustrated) embodiments, however, the cylindrical wall 12 may extend downward from the base 11, such that the top surface 16 of the cylindrical wall 12 is part of the upper surface of the base. In some embodiments, the syringe nest 10 may be configured to hold syringe barrels having volumes of 1 mL or higher, for example syringe barrels having volumes between 1 mL and 3 mL.

The syringe nest 10 also comprises one or more ribs 20 positioned around the upper opening 15 of each receptacle 12, e.g. on the top surface 16 of the cylindrical wall 12. As shown in FIGS. 2-3, the one or more ribs 20 extend upward from the top surface 16 of the cylindrical wall 12. The specific height of the one or more ribs 20 is only important in that the height be sufficient to provide abutting contact with the outer surface of the rear flange 103 of the syringe barrel 100 but not so high as to interfere with processing to which the syringe nest 10 is to be subjected, e.g. filling and stoppering processes, sealing of a tub containing the nest, etc.

In the illustrated embodiment, the one or more ribs 20 is made up of a pair of ribs, each of which extends around slightly less than half of the circumference of the upper opening 15 of the receptacle 12, such that a small gap is formed between the first and second ribs. In other (non-illustrated) embodiments, the one or more ribs 20 may be a single rib that extends around the full circumference of the upper opening 15 of the receptacle 12. In yet other (non-illustrated) embodiments, the one or more ribs 20 may comprise any number of ribs, e.g. three ribs, four ribs, five ribs, six ribs, etc., positioned at intervals around the circumference of the upper opening 15 of the receptacle 12.

The one or more ribs 20 is configured to limit radial movement of a syringe barrel 100 within the receptacle by providing abutting contact between the one or more ribs and an outer surface of the syringe barrel rear flange 103. By doing so, the one or more ribs 20 are configured to keep a syringe barrel 100 substantially centered within the receptacle 12.

Desirably, the one or more ribs 20 may be positioned to provide an inner, abutting contact surface having a diameter that is close to (but larger than) the diameter of the rear flange 103 of a particular type of syringe barrel 100, e.g. for which the syringe nest 10 is specifically designed.

In some embodiments, for instance, the one or more ribs 20 may be configured to be positioned 1.5 mm or less from the outer surface of the rear flange 103 when a syringe barrel is centered within the receptacle 12 (meaning that the diameter of the one or more ribs, as measured from one side of the upper opening 15 of the receptacle to an opposite side of the upper opening of the receptacle, may be within 3.0 mm of the diameter of the rear flange, D3); alternatively the one or more ribs may be configured to be positioned 1.25 mm or less from the outer surface of the rear flange 103 when a syringe barrel is centered within the receptacle 12 (meaning that the diameter of the one or more ribs, as measured from the inner surface of the rib on one side of the upper opening of the receptacle to the inner surface of the rib on an opposing side of the upper opening of the receptacle, may be within 2.5 mm of the diameter of the rear flange, D3); alternatively the one or more ribs may be configured to be positioned 1.0 mm or less from the outer surface of the rear flange 103 when a syringe barrel is centered within the receptacle 12 (meaning that the diameter of the one or more ribs, as measured from one side of the upper opening of the receptacle to an opposite side of the upper opening of the receptacle, may be within 2.0 mm of the diameter of the rear flange, D3); alternatively the one or more ribs may be configured to be positioned 0.75 mm or less from the outer surface of the rear flange 103 when a syringe barrel is centered within the receptacle 12 (meaning that the diameter of the one or more ribs, as measured from one side of the upper opening of the receptacle to an opposite side of the upper opening of the receptacle, may be within 1.5 mm of the diameter of the rear flange, D3); alternatively the one or more ribs may be configured to be positioned 0.5 mm or less from the outer surface of the rear flange 103 when a syringe barrel is centered within the receptacle 12 (meaning that the diameter of the one or more ribs, as measured from one side of the upper opening of the receptacle to an opposite side of the upper opening of the receptacle, may be within 1.0 mm of the diameter of the rear flange, D3).

In these embodiments, therefore, the radial, i.e. lateral, movement of the syringe barrel may be limited to 1.5 mm or less from a central position, alternatively 1.25 mm or less from a central position, alternatively 1.0 mm or less from a central position, alternatively 0.75 mm or less from a central position, alternatively 0.50 mm or less from a central position.

In some embodiments, depending on the surface area of the top surface 16 of the cylindrical wall that supports the rear flange 103 of the syringe barrel 100, the one or more ribs 20 may be positioned such that the inner surface of the one or more ribs is between about 0.50 mm and about 2.0 mm from the upper opening 15 of the receptacle 12, alternatively between about 0.75 mm and about 1.75 mm from the upper opening of the receptacle, alternatively between about 1.0 mm and about 1.5 mm from the upper opening of the receptacle.

In some embodiments, the difference between D4 and D1 may be at least 4 mm, meaning that absent the one or more ribs 20 of the present disclosure, the syringe barrel 100 would be able to move radially 2 mm or more in any direction from a central position, i.e. the gap, G, is at least 2 mm. In some embodiments, the difference between D4 and D1 may be at least 5 mm, meaning that absent the one or more ribs 20 of the present disclosure, the syringe barrel 100 would be able to move radially 2.5 mm or more in any direction from a central position, i.e. the gap, G, is at least 2.5 mm. For example, in some embodiments, the difference between D4 and D1 may be between about 4 mm and about 7 mm, meaning that absent the one or more ribs 20 of the present disclosure, the syringe barrel 100 may be able to move radially between about 2 mm and about 3.5 mm in any direction from a central position, i.e. the gap, G, is between about 2 mm and about 3.5 mm.

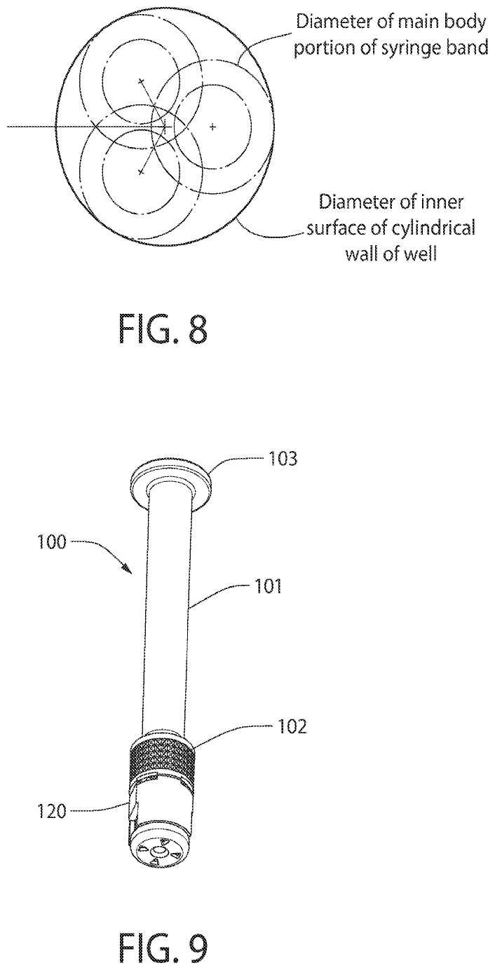

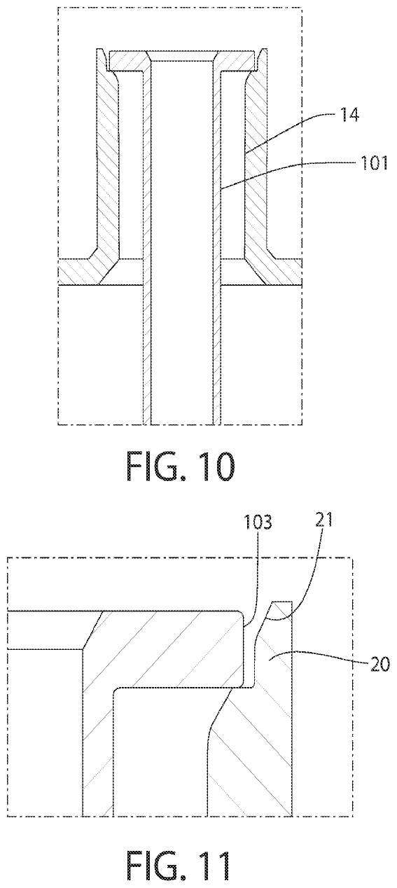

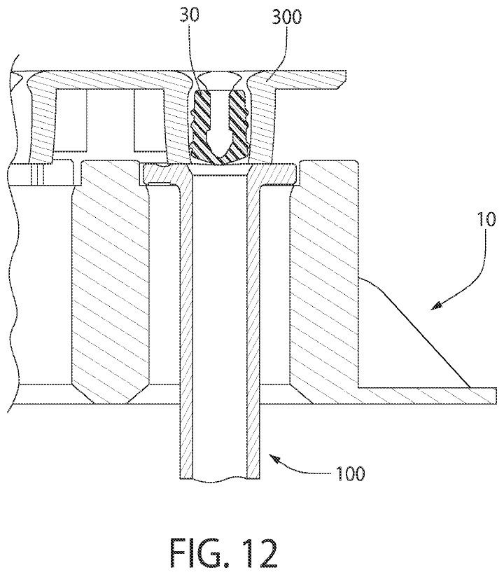

An example of syringe barrel movement that may occur absent the ribs 20 of the present disclosure is illustrated in FIG. 8. As shown in FIG. 8, the main body portion 101 of a syringe barrel held within a receptacle 12 of a syringe nest may be able to move from a central position in any radial direction (three examples of which are shown). Such radial movement of the syringe barrel 101 could result in the filling needle used during a filling process, which is desirably aligned with the center of the receptacle, contacting a portion of the syringe barrel (e.g. top surface of the flange 103, inner surface of the wall defining the lumen, etc.), which could result in improper filling and/or damage to the syringe barrel, the filling equipment, or both. Similarly, a high amount of radial movement of the syringe barrel 101 could result in a plunger contacting a portion of the syringe barrel (e.g. top surface of the flange 103) during a stoppering process, which could have similar undesirable effects, e.g. a failure to stopper the filled syringe barrel and/or damage to the syringe barrel, the stoppering equipment, or both. Less drastic radial movement of the syringe barrel 101 within the receptacle 12 could lead to the plunger being inserted into the lumen of the syringe barrel but in a manner where an adequate seal and thus container closure integrity (CCI) is not obtained.

Where the one or more ribs 20 are positioned to provide an inner surface that is close to the outer surface of the rear flange 103 of a syringe barrel 100 inserted in the receptacle 12, it may be desirable to provide the one or more ribs 20 with a chamfered or bevelled surface 21 that is configured to direct the flange of the syringe barrel toward the upper opening 15 of the receptacle during insertion of the syringe barrel into the receptacle. An example of such a chamfered surface 21 is shown in FIG. 2. By providing a sloped transition between the upper surface and the inner surface of each of the one or more ribs 20, if a syringe barrel 100 is not perfectly aligned with the receptacle 12 when being inserted, the rear flange 103 of a syringe barrel will come into contact with the chambered surface 21 and the syringe barrel directed into better alignment with the receptacle.

Embodiments of the syringe nest 10 of the present disclosure may be used to hold a plurality of syringe barrels 100 during a method of preparing a plurality of pre-filled syringes, including for instance during a filling step, a stoppering step, or both. By reducing radial movement of the syringe barrels 100 within the receptacles 12 of the syringe nest 10, i.e. by keeping each of the syringe barrels substantially centered in a receptacle, the ribs 20 help ensure that the filling inserts (also referred to as filling needles), and more importantly the plungers 130-given the tight dimensional tolerances between a plunger and the inner wall of the syringe barrel 100 into which the plunger is inserted-are properly aligned with the plurality of syringe barrels during the filling and/or stoppering process.

Accordingly, embodiments of the present disclosure are directed to a method of preparing a plurality of pre-filled syringes. The method may include a filling step, a stoppering step, or both, in which a plurality of syringe barrels 100 are positioned within receptacles 12 of a syringe nest 10 in accordance with an embodiment of the present disclosure. Each of the syringe barrels 100 may include a removable tip cap 120 whose diameter is greater than the diameter of the main body portion 101 of the syringe barrel. And the syringe nest 10 may comprise, around the upper opening 15 of each receptacle 12, one or more ribs 20 configured to keep each syringe barrel substantially stable, i.e. having limited movement in a radial direction, and centered within the receptacle.

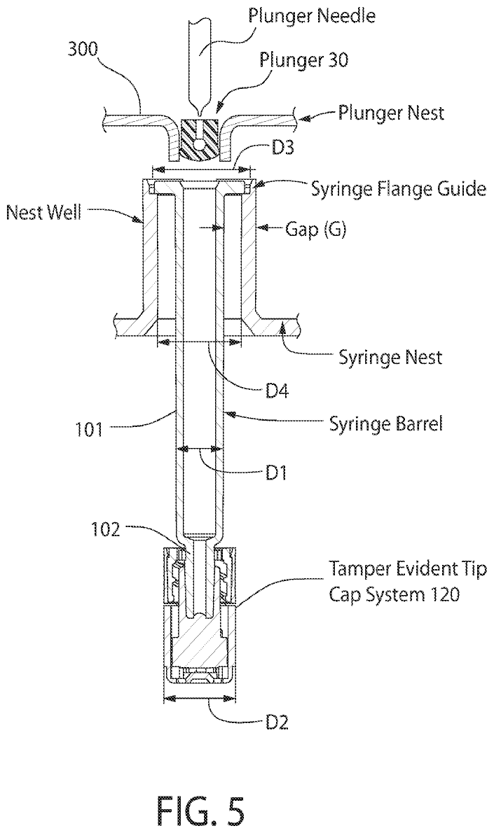

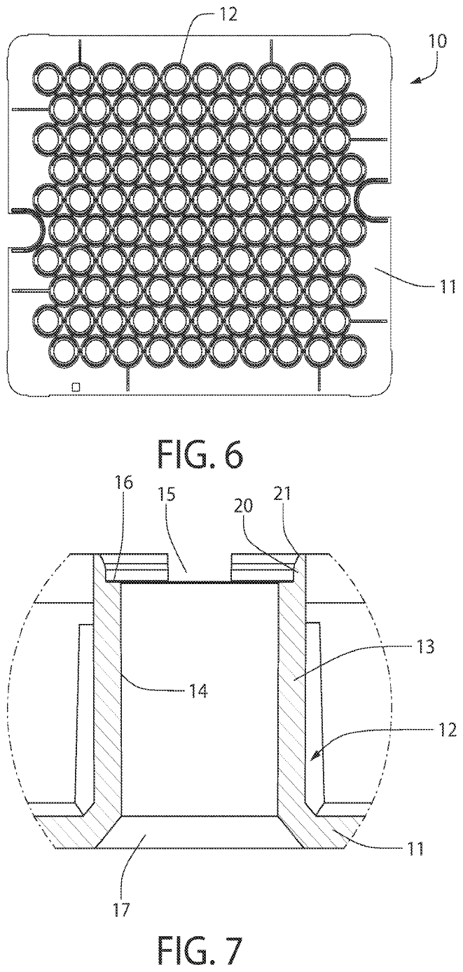

In some embodiments, the method may comprise a step of inserting a plurality of plungers into the lumens of a plurality of syringe barrels, i.e. a stoppering step. An example of a system that may be employed in a stoppering step is shown, for example, in FIGS. 4-5. In such a step, a plurality of syringe barrels 100 are held in the receptacles 12 of an embodiment of a syringe nest 10 as disclosed herein, and a plurality of plungers 30 are held in a plunger nest 300. The syringe nest 10 and the plunger nest 300 are aligned so that each plunger 30 is positioned substantially directly above a rear opening 105 of a syringe barrel 100, e.g. as shown in FIG. 5. The one or more ribs 20 surrounding each receptacle 12 of the syringe nest 10 ensure that each of the plurality of syringe barrels 100 is substantially centered in a receptacle of the syringe nest and therefore positioned to receive the plunger 30. A fixture comprising a plurality of plunger insertion rods 400 is then operated so as to push each plunger 30 out of the plunger nest 300 and into a lumen 104 of a syringe barrel 100 held within the syringe nest 10.

In some embodiments, the method may comprise a step of filling the lumens of a plurality of syringe barrels with an injectable medical solution. In such a step, a plurality of syringe barrels 100 are held in the syringe nest 10 as disclosed herein. The syringe nest 10 is then aligned with a fixture comprising a plurality of filling needles, with a filling needle being positioned substantially directly above a rear opening 105 of each syringe barrel 100. The one or more ribs 20 surrounding each receptacle 12 of the syringe nest 10 ensure that each of the plurality of syringe barrels 100 is substantially centered in a receptacle of the syringe nest and therefore correctly positioned to receive the filling needle. The plurality of filling needles are then inserted into the lumens 104 of the syringe barrels 100 through the rear openings 105, and an injectable medical solution is injected from the filling needles into the lumens of the plurality of syringe barrels until a desired volume of solution is present in the lumen. The filling needles are then removed from the lumens 104 of the syringe barrels 100 and the syringe nest 10 may be transported to a stoppering station where the syringe barrels may be stoppered, e.g. as described herein.

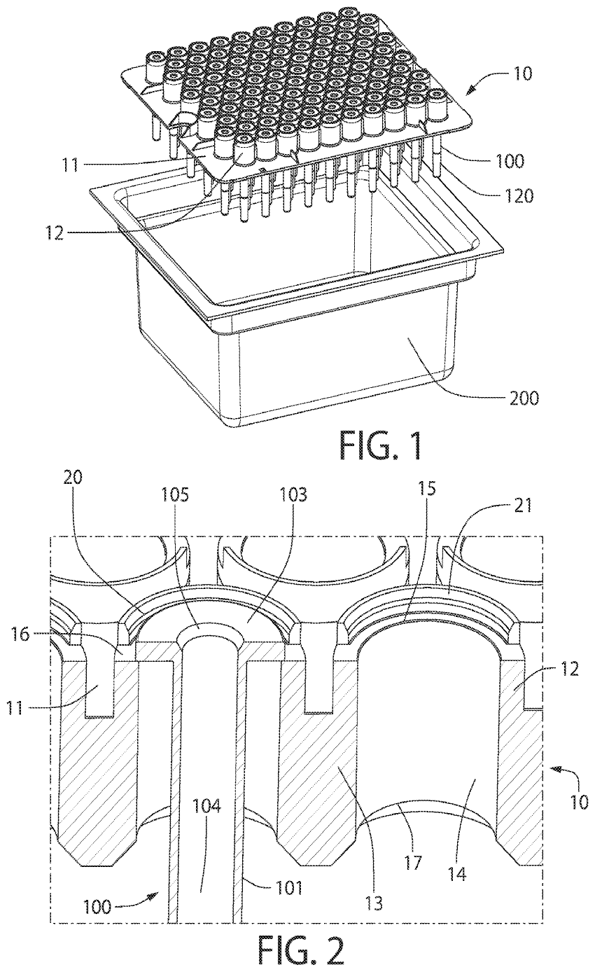

Embodiments of the present disclosure are also directed to packages of pre-filled syringes, in which the pre-filled syringes are positioned within receptacles 12 of a syringe nest 10 according to an embodiment of the present disclosure. An example of such a package is shown in FIG. 1. The package may comprise a syringe nest 10 as described herein, a plurality of pre-filled syringes, and a tub 200 in which the syringe nest is held. The tub 200 may comprise a shoulder on which the syringe nest 10 is seated so that the syringe nest 10 and the syringes contained therein are positioned within an interior of the tub, as defined by the sidewalls of the tub. Typically, the syringes may be suspended above the bottom wall of the tub. The package may also include a removable cover, such as a flexible membrane sealed to an upper flange of the tub. The tub may be configured in accordance with ISO 11040-7.

In some embodiments, the nest 10 may be made of plastic. For instance, the nest 10 may be made of polypropylene. In some embodiments, the nest 10 may be a single, integral piece. For example, in some embodiments, the nest 10 may be injection molded from a plastic material, e.g. polypropylene.

In some embodiments, the nest 10 may comprise one hundred receptacles, e.g. in ten rows of ten.

### Example Embodiments

1. A syringe nest comprising:
a substantially planar base; and
a plurality of syringe receptacles, each of the receptacles including a cylindrical wall defining an upper opening;
   wherein each of the receptacles is sized and configured to receive a syringe barrel having a main body portion, a front dispensing portion, and a rear flange;
   wherein an inner surface of the cylindrical wall has a diameter that is larger than the diameter of the main body portion of the syringe barrel but smaller than the diameter of the rear flange; and
one or more upward-extending ribs positioned around the upper opening of each receptacle;
wherein the one or more ribs are configured to limit radial movement of a syringe barrel within the receptacle through contact between the one or more ribs and an outer surface of the syringe flange.

### 2. a Syringe Nest Comprising:

a substantially planar base; and
a plurality of syringe receptacles, each of the receptacles including a cylindrical wall defining an upper opening;
   wherein each of the receptacles is sized and configured to receive a syringe barrel having a main body portion, a front dispensing portion, and a rear flange;
   wherein an inner surface of the cylindrical wall has a diameter that is larger than the diameter of the main body portion of the syringe barrel but smaller than the diameter of the rear flange; and
one or more upward-extending ribs positioned around the upper opening of each receptacle;

wherein the one or more ribs are configured to maintain each of the plurality of syringe barrels substantially centered within the receptacle.

3. The syringe nest of any preceding embodiment, further comprising a syringe barrel having a main body portion, a front dispensing portion, and a rear flange positioned within each receptacle.
a substantially Planar Base; and
a plurality of syringe receptacles, each of the receptacles including a cylindrical wall defining an upper opening;
a plurality of syringe barrels, each syringe barrel being removably positioned within one of the plurality of receptacles, and each syringe barrel having a main body portion, a front dispensing portion, and a rear flange;
one or more upward-extending ribs positioned around the upper opening of each receptacle;
wherein an inner surface of the cylindrical wall has a diameter that is larger than the diameter of the main body portion of the syringe barrel but smaller than the diameter of the rear flange; and
wherein the one or more ribs limit radial movement of the syringe barrel within the receptacle through contact between the one or more ribs and an outer surface of the syringe flange.
   5. A syringe nest comprising:
a substantially planar base; and
a plurality of syringe receptacles, each of the receptacles including a cylindrical wall defining an upper opening;
a plurality of syringe barrels, each syringe barrel being removably positioned within one of the plurality of receptacles, and each syringe barrel having a main body portion, a front dispensing portion, and a rear flange;
one or more upward-extending ribs positioned around the upper opening of each receptacle;
wherein an inner surface of the cylindrical wall has a diameter that is larger than the diameter of the main body portion of the syringe barrel but smaller than the diameter of the rear flange; and
wherein the one or more ribs maintain each of the plurality of syringe barrels substantially centered within the receptacle.
   6. The syringe nest of any preceding embodiment, wherein the one or more ribs comprises a single rib that extends around the circumference of the upper opening of the receptacle.
   7. The syringe nest of any preceding embodiment, wherein the one or more ribs comprises two or more ribs, each of which extends around a portion of the circumference of the upper opening of the receptacle.
   8. The syringe nest of any preceding embodiment, wherein at least a portion of the cylindrical wall of the receptacle extends upward from the base, and wherein the one or more ribs extend from a top surface of the cylindrical wall.
   9. The syringe nest of any preceding embodiment, wherein the upper opening of the receptacle is substantially planar with an upper surface of the base and wherein the one or more ribs extends upward from the upper surface of the base.
   10. The syringe nest of any preceding embodiment, wherein the cylindrical wall of each receptacle also defines a lower opening.
   11. The syringe nest of any preceding embodiment, wherein the one or more ribs are configured to have an inner surface positioned 1.5 mm or less, alternatively 1.25 mm or less, alternatively 1.0 mm or less, alternatively 0.75 mm or less, alternatively 0.50 mm or less from the outer surface of the flange when a syringe barrel is centered within the receptacle.
   12. The syringe nest of any preceding embodiment, wherein the one or more ribs have an inner surface positioned 1.5 mm or less, alternatively 1.25 mm or less, alternatively 1.0 mm or less, alternatively 0.75 mm or less, alternatively 0.50 mm or less from the flange when the syringe barrel is centered within the receptacle.
   13. The syringe nest of any preceding embodiment, wherein the one or more ribs provide an inner surface positioned between about 0.50 mm and about 2.0 mm from the upper opening of the receptacle, alternatively between about 0.75 mm and about 1.75 mm from the upper opening of the receptacle, alternatively between about 1.0 mm and about 1.5 mm from the upper opening of the receptacle.
   14. The syringe nest of any preceding embodiment, wherein the diameter of the inner surface of the cylindrical wall is at least 4 mm larger than the diameter of the main body portion of the syringe barrel, alternatively at least 5 mm larger than the diameter of the main body portion of the syringe barrel.
   15. The syringe nest of any preceding embodiment, wherein each of the one or more ribs comprises a chamfered surface configured to direct the flange of a syringe barrel toward the upper opening of the receptacle during insertion of the syringe barrel into the receptacle.
   16. The syringe nest of any preceding embodiment, wherein the syringe comprises a staked needle and a removable needle shield.
   17. The syringe nest of any preceding embodiment, wherein the needle shield is tamper-evident.
   18. The syringe nest of any preceding embodiment, wherein the needle shield has a diameter that is greater than the diameter of the main portion of the body of the syringe and less than the diameter of the rear flange.
   19. The syringe nest of any preceding embodiment, wherein the syringe comprises a Luer tip and a removable tip cover.
   20. The syringe nest of any preceding embodiment, wherein the tip cover is tamper-evident.
   21. The syringe nest of any preceding embodiment, wherein the tip cover has a diameter that is greater than the diameter of the main portion of the body of the syringe and less than the diameter of the rear flange.
   22. The syringe nest of any preceding embodiment, wherein the nest is made of plastic, optionally wherein the nest is made of polypropylene, optionally wherein the nest is injection molded.
   23. The syringe nest of any preceding embodiment, wherein the nest comprises between fifty and one hundred receptacles.
   24. The syringe nest of any preceding embodiment, wherein the syringe barrels have a volume of 1 mL or less, optionally less than 1 mL, optionally 0.75 mL or less, optionally less than 0.75 mL, optionally 0.5 mL or less, optionally wherein the syringe barrels have a volume of 0.5 mL.
   25. A set of nested syringe barrels comprising:

the syringe nest of any preceding embodiment; and a plurality of syringes barrels, each of the plurality of syringe barrels being received within one of the plurality of receptacles.

26. A set of nested syringes comprising:
the syringe nest of any preceding embodiment; and a plurality of syringes, each of the plurality of syringes having a syringe barrel received within one of the plurality of receptacles;
wherein each of the plurality of syringes further comprises
   an injectable medical solution in a lumen of the syringe barrel,
   a removable needle shield or a removable tip cap sealing a dispensing opening, and
   a plunger sealing a rear opening of the lumen.
      27. A package of syringes comprising:
the syringe nest of any preceding embodiment; and
   a plurality of syringes, each of the plurality of syringes having a syringe barrel received within one of the plurality of receptacles;
a tub comprising a shoulder on which the syringe nest is seated such that the syringes are positioned within an interior of the tub defined by the tub sidewalls; and
a cover sealed to the tub to enclose the syringes therein;
wherein each of the plurality of syringes comprises:
   an injectable medical solution in a lumen of the syringe barrel,
   a removable needle shield or a removable tip cap sealing a dispensing opening, and
   a plunger sealing a rear opening of the lumen.
      28. The package of syringes of any preceding embodiment, wherein the cover is a flexible membrane
      29. A method of preparing a plurality of pre-filled syringes, the method comprising a step of inserting a plurality of plungers into the lumens of a plurality of syringe barrels, in which:
a plurality of syringe barrels are held in the syringe nest of any preceding embodiment,
a plurality of plungers are held in a plunger nest,
the plunger nest is aligned with the syringe nest, and
a fixture comprising a plurality of plunger insertion rods pushes the plurality of plungers out of the plunger nest and a desired distance into the lumens of the syringe barrels.
   30. A method of preparing a plurality of pre-filled syringes, the method comprising a step of filling the lumens of a plurality of syringe barrels with an injectable medical solution, in which:
a plurality of syringe barrels are held in the syringe nest of any preceding embodiment,
a plurality of filling needles are inserted into the lumens of the syringe barrels, and
an injectable medical solution is injected from the filling needles into the lumens of the plurality of syringe barrels.
   31. The method of any preceding embodiment, wherein the one or more ribs ensure that each of the plurality of syringe barrels is substantially centered in a receptacle of the syringe nest.
   32. A method of preparing a plurality of pre-filled syringes, the method comprising:
(i) filling the lumens of a plurality of syringe barrels with an injectable medical solution, in which:
   a plurality of syringe barrels are held in the syringe nest of any preceding embodiment,
   a plurality of filling needles are inserted into the lumens of the syringe barrels, and
   an injectable medical solution is injected from the filling needles into the lumens of the plurality of syringe barrels; and
(ii) inserting a plurality of plungers into the lumens of a plurality of syringe barrels, in which:
the plurality of syringe barrels are held in the syringe nest,
a plurality of plungers are held in a plunger nest,
the plunger nest is aligned with the nest, and
a fixture comprising a plurality of plunger insertion rods pushes the plurality of plungers out of the plunger nest and into the lumens of the syringe barrels;
wherein the one or more ribs ensure that each of the plurality of syringe barrels is substantially centered in a receptacle of the nest during both the filling and plunger insertion steps.

It can be seen that the described embodiments provide a unique and novel syringe nest that has a number of advantages over those in the art. While there is shown and described herein certain specific structures embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

1. A syringe nest comprising:
a substantially planar base; and
a plurality of syringe receptacles, each of the receptacles including a cylindrical wall defining an upper opening;
wherein each of the receptacles is sized and configured to receive a syringe barrel having a main body portion, a front dispensing portion, and a rear flange;
wherein an inner surface of the cylindrical wall has a diameter that is larger than the diameter of the main body portion of the syringe barrel but smaller than the diameter of the rear flange; and
one or more upward-extending ribs positioned around the upper opening of each receptacle;
wherein the one or more ribs are configured to limit radial movement of a syringe barrel within the receptacle through contact between the one or more ribs and an outer surface of the syringe flange.

2. The syringe nest of claim 1, further comprising:
a plurality of syringe barrels, each syringe barrel being removably positioned within one of the plurality of receptacles, and each syringe barrel having a main body portion, a front dispensing portion, and a rear flange.

3. The syringe nest of claim 1, wherein the one or more ribs are configured to maintain the syringe barrels substantially centered within the receptacle.

4. The syringe nest of claim 1, wherein the one or more ribs comprises a single rib that extends around the circumference of the upper opening of the receptacle.

5. The syringe nest of claim 1, wherein the one or more ribs comprises two or more ribs, each of which extends around a portion of the circumference of the upper opening of the receptacle.

6. The syringe nest of claim 1, wherein at least a portion of the cylindrical wall of the receptacle extends upward from the base, and wherein the one or more ribs extend from a top surface of the cylindrical wall.

7. The syringe nest of claim 1, wherein the upper opening of the receptacle is substantially planar with an upper surface of the base and wherein the one or more ribs extends upward from the upper surface of the base.

8. The syringe nest of claim 1, wherein the one or more ribs are configured to have an inner surface positioned 1.5 mm or less, from the outer surface of the flange when a syringe barrel is centered within the receptacle.

9. The syringe nest of claim 1, wherein the one or more ribs provide an inner surface positioned between about 0.50 mm and about 2.0 mm from the upper opening of the receptacle.

10. The syringe nest of claim 1, wherein the diameter of the inner surface of the cylindrical wall is at least 4 mm larger than the diameter of the main body portion of the syringe barrel.

11. The syringe nest of claim 1, wherein each of the one or more ribs comprises a chamfered surface configured to direct the flange of a syringe barrel toward the upper opening of the receptacle during insertion of the syringe barrel into the receptacle.

12. The syringe nest of claim 2, wherein each of the plurality of syringes comprises a staked needle and a removable needle shield, the needle shield having a diameter that is greater than the diameter of the main body portion of the syringe and less than the diameter of the rear flange of the syringe.

13. The syringe nest of claim 2, wherein each of the plurality of syringes comprises a Luer tip and a removable tip cover, the tip cover having a diameter that is greater than the diameter of the main body portion of the syringe and less than the diameter of the rear flange of the syringe.

14. The syringe nest of claim 2, wherein each of the plurality of syringe barrels has a volume of 1 mL or less.

15. A set of nested syringes comprising:
the syringe nest of claim 1; and
a plurality of syringes, each of the plurality of syringes having a syringe barrel received within one of the plurality of receptacles;
wherein each of the plurality of syringes further comprises
an injectable medical solution in a lumen of the syringe barrel,
a removable needle shield or a removable tip cap sealing a dispensing opening, and
a plunger sealing a rear opening of the lumen.

16. A package of syringes comprising:
the syringe nest of claim 1; and
a plurality of syringes, each of the plurality of syringes having a syringe barrel received within one of the plurality of receptacles;
a tub comprising a shoulder on which the syringe nest is seated such that the syringes are positioned within an interior of the tub defined by the tub sidewalls; and
a cover sealed to the tub to enclose the syringes therein;
wherein each of the plurality of syringes comprises:
an injectable medical solution in a lumen of the syringe barrel,
a removable needle shield or a removable tip cap sealing a dispensing opening, and
a plunger sealing a rear opening of the lumen.

17. A method of preparing a plurality of pre-filled syringes, the method comprising a step of inserting a plurality of plungers into the lumens of a plurality of syringe barrels, in which:
a plurality of syringe barrels are held in the syringe nest of any preceding claim 1,
a plurality of plungers are held in a plunger nest,
the plunger nest is aligned with the syringe nest, and
a fixture comprising a plurality of plunger insertion rods pushes the plurality of plungers out of the plunger nest and a desired distance into the lumens of the syringe barrels.

18. A method of preparing a plurality of pre-filled syringes, the method comprising a step of filling the lumens of a plurality of syringe barrels with an injectable medical solution, in which:
a plurality of syringe barrels are held in the syringe nest of claim 1,
a plurality of filling needles are inserted into the lumens of the syringe barrels, and
an injectable medical solution is injected from the filling needles into the lumens of the plurality of syringe barrels.

19. A method of preparing a plurality of pre-filled syringes, the
(i) filling the lumens of a plurality of syringe barrels with an injectable medical solution, in which:
a plurality of syringe barrels are held in the syringe nest of claim 1.
a plurality of filling needles are inserted into the lumens of the syringe barrels, and
an injectable medical solution is injected from the filling needles into the lumens of the plurality of syringe barrels; and
(ii) inserting a plurality of plungers into the lumens of a plurality of syringe barrels, in which:
the plurality of syringe barrels are held in the syringe nest,
a plurality of plungers are held in a plunger nest,
the plunger nest is aligned with the nest, and
a fixture comprising a plurality of plunger insertion rods pushes the plurality of plungers out of the plunger nest and into the lumens of the syringe barrels;
wherein the one or more ribs ensure that each of the plurality of syringe barrels is substantially centered in a receptacle of the nest during both the filling and plunger insertion steps.

20. The method of any preceding claim 17, wherein the one or more ribs ensure that each of the plurality of syringe barrels is substantially centered in a receptacle of the syringe nest.
